(19) United States
(12) Patent Application Publication
(10) Pub. No.: US 2018/0104109 A1
(43) Pub. Date: Apr. 19, 2018

(54) DRESSING FOR PUNCTURE OR INJECTION SITE, PARTICULARLY FOR HEMODIALYSIS

(71) Applicant: NEPHROKIT, Le Vesinet (FR)

(72) Inventor: Mokhtar CHAWKI, Le Vesinet (FR)

(73) Assignee: NEPHROKIT, Le Vesinet (FR)

(21) Appl. No.: 15/684,039

(22) Filed: Aug. 23, 2017

Related U.S. Application Data
(60) Provisional application No. 62/408,322, filed on Oct. 14, 2016.

(51) Int. Cl. A61F 13/02; A61L 15/24; A61L 15/58; A61L 15/42

(52) U.S. Cl. CPC A61F 13/025; A61F 13/0206; A61L 15/425; A61L 15/24; A61L 15/58; A61F 13/0266

(57) ABSTRACT

Dressing (10) for puncture or injection site, comprising a sheet of micro-perforated polymer material, a first face of said sheet being covered with a pressure-sensitive adhesive, the opposite face of said sheet being non-adhesive, characterized in that it comprises a strip of material attached to the non-adhesive face of the sheet of polymer material, said strip of material being compressible and impermeable.

The invention relates to the medical or veterinary domain.

More particularly, the invention concerns a dressing for a puncture site or injection site.

“Puncture” as used here designates the operation by which a substance, generally fluid, is evacuated from a part of the body of a human or animal.

An example of puncture is blood collection, a common procedure enabling laboratory examinations of a blood sample taken by venous, capillary or arterial puncture. The hypodermic needle used for removing blood is often connected to vacuum tubes (for example the Vacutainer®, marketed by the Becton Dickinson company).

“Injection” as used here designates the operation by which a substance, generally fluid, is introduced into a cavity or into the vessels of the body of a human or animal.

An example of injection is intravenous perfusion, a common technique of parenteral injection for the drip administration of drugs, fluids or blood products into the veins, generally a peripheral vein of an upper limb. In particular, intravenous perfusion enables the administration of solutes in order to correct blood volume.

“Puncture site or injection site” here designates an area of skin of the body of a human or of an animal through which a puncture or injection device passes (in particular a needle, a catheter, or a cannula).

“Dressing for a puncture or injection site” here designates a protective device covering the wound formed by the puncture or injection site.

More particularly, the invention relates to the medical or veterinary use of a vascular puncture or injection site, for example needle/catheter perfusions or short intravenous devices such as Cathlon® (PTFE cannulas).

Still more particularly, the invention relates to the domain of hemodialysis.

It is common to remove or inject fluids or administer drugs to a patient or an animal by means of a tube attached to a needle or a catheter. It is estimated that about 80% of hospitalized patients receive treatment administered by intravenous catheter.

By puncture or injection, a device is inserted through the skin (hypodermic needle, catheter, or cannula).

For example, butterfly needles are employed for transfusions, particularly for drawing blood. These needles are so named because they are provided with a manual pinching area in the form of butterfly wings. Folding these wings together enables a good grip and facilitates insertion of the needle beneath the skin. Once the needle is in place, the wings normally rest on the skin and adhesive tape is placed on the wings and on the skin to prevent extraction of the needle. Examples of butterfly needles can be found in the American patents issued under the following numbers: U.S. Pat. Nos. 2,725,058, 3,064,648, 3,640,275, 4,194,504, 4,300,553, 4,627,842, 5,108,376, 5,149,328, 6,270,480.

Some patients require chronic use of peripheral venous catheters. This is the case in particular for patients suffering from acute or chronic renal insufficiency treated by hemodialysis, or extra-renal purification.

Three types of vascular access predominate for hemodialysis: arteriovenous fistulas (AVF), arteriovenous prostheses or grafts, and central venous catheters (CVC).

AVFs are anastomoses created surgically to connect an artery and a vein of the patient, commonly in the forearm or arm, most often between a radial or humeral artery and its homonymous vein. This anastomosis makes it possible to increase the blood flow in that vein.

The needle taking the blood from the patient to the dialysis apparatus is called artery, while the one returning the blood to the patient is called vein.

When the needle is removed from a blood vessel or a dialysis fistula, several risks must be taken into account: hemorrhage, blood spray, contamination of healthcare personnel, hematoma, bacterial contamination, [etc].

The risk of hemorrhage is very high for certain patients suffering from acute or chronic renal insufficiency treated by hemodialysis. During hemodialysis, an anticoagulant is often employed to limit the risks of blockage of the capillaries by thrombosis. At the end of the dialysis session, any hemorrhage from the puncture site may prove fatal for the patient.

The risks of blood spray and contamination are significant. The spread of AIDS and hepatitis, among other diseases transmissible by blood, only makes this risk more dreaded. According to a circular from the General Health Department dated Apr. 20, 1998 (which can be viewed at http://www.sante.gouv.fr) concerning the prevention of transmission of infectious agents carried by blood or biological fluids during treatment in healthcare facilities, accidental exposure to blood (AEB) is defined as “any contact with blood or a biological fluid containing blood and involving either a break in the skin or spray on a mucosal surface (eye) or on damaged skin.” The risk of transmission of infectious diseases during an AEB in hemodialysis is currently highest from the hepatitis C virus because of its prevalence in hemodialysis patients. The risk of transmission of HIV after exposure to blood of a patient carrying HIV is estimated on average at 0.32%. The risk of transmission of the BHV from an infected patient is very high, between 2% and 40%. This high degree of contagiousness is related to the very large quantity of virus present in blood and biological fluids (between 1 million and 1 billion viral particles per milliliter). According to the RAISIN report (monitoring accidents of exposure to blood in French healthcare facilities in 2005, available at http://www.invs.sante.fr), the majority of accidental exposures to blood occur at the time of removal of a needle from the skin. According to the report from the AEB-Raisin network, for the years 2013-2014 the rate of AEBs per 100 beds is about 6%, and 20% of AEBs result from a spray, particularly when withdrawing a needle from a patient's skin.

For hemodialysis sessions on AVF, the time of greatest risk of contamination by blood spray is during compression, primarily at the time the needle is withdrawn. Indeed, the hemostatic compression bandage can be improperly positioned on the puncture site, in particular for AVFs having a strong flow or aneurysmal areas.

At the end of a dialysis session, during disconnection, there is a risk of bacterial contamination, generally staphylococcal. This risk is elevated at the time of compression of the puncture sites or during a recurrence of bleeding away from the dialysis session.

The wound formed during each dialysis session has specific characteristics.

First, this wound occurs repeatedly in a limited area, with large diameter needles. A hemodialysis session lasts about four hours, and must be done three times per week. The needles used to puncture the AVFs are large gauge, with an inside diameter typically varying between 1.6 and 2 mm.

Secondly, the skin surrounding the puncture or injection site in the AVF is in principle non-exudative and non-secretive because only the blood can be present; post-dialysis bleeding being a known complication. Some patients remain at risk even after manual compression of 30 minutes (Perera et al, A novel use of 2 octyl-cyanocrylate controlling post hemodialysis site hemorrhage, The Journal of Emergency Medicine, 44 vol 2, pp. 467-468 2013). Hemostasis is obtained within a variable period of time, on the order of 15 to 20 minutes according to Schwab et al, Prevention of hemodialysis fistula thrombosis. Early detection of venous stenosis, Kidney International, 36, pp. 707-711 (1989), and could be reduced to about three minutes by local administration of thrombin according to Varizi, Topical thrombin and control of bleeding from the fistula puncture sites in dialyzed patients, Nephron 24, pp. 254-256 (1979). The use of poly-β-1→4-N-acetyl glucosamine would enable compression times on the order of 1 to 14 minutes (U.S. Pat. No. 8,992,453, page 4 lines 44-54). In dialyzed patients with an AVF with high blood flow, the effect of anticoagulants is such that the bleeding time at the puncture site can be up to 30 minutes (see WO 2004/060245 page 1, third paragraph) and even 45 minutes, after the dialysis session (see WO 03/099143, page 1 lines 11-20). There is a large number of patents and patent applications for compression dressings, attempting to provide a solution to these bleeding times (see for example US 2004/0092999, WO 99/08723, US 2005/0256438, WO 2007/044647, WO 03/099143, U.S. Pat. No. 5,891,074, US 20060155235, U.S. Pat. No. 3,490,448, U.S. Pat. No. 6,316,686).

Thirdly, the wound occurs in a deformed area. The creation of an AVF actually strongly modifies the appearance of the forearm of the patient by creating aneurysmal areas.

Moreover, dialysis patients are frequently elderly, with the consequences resulting from skin sensitivity to dressings.

The means used in hemodialysis units to limit the risks of AEB and hemorrhage are not standardized.

In particular, disconnection protocols are not uniform, taking into account several parameters: autonomy of the patients, physical capability of the patients enabling them to be more or less involved in their care, fragility of the skin of patients who for the most part are elderly, whether or not there are aneurysmal areas, taking anticoagulant drugs of the acetylsalicylic acid type or anti-vitamin K or anti-inflammatories.

The flow rate of the AVF as well as the presence of hyper pressure by stenosis of the vascular access can combine to prolong the compression time.

Known from the prior art are several techniques to avoid the risks of AEB and hemorrhage that have just been described.

The means for obtaining hemostasis on an AVF can be classified in four categories:
manual compression,
mechanical devices comprising a spring mechanism (fistula pressure clamp), see for example CN 203169258,
tape providing compression by tightening,
pads comprising a compress loaded with a hemostatic agent (gelatin, collagen, chitosan, cellulose oxide, kaolin, zeolite), see for example Bachtell et al, Treatment of dialysis access puncture wound bleeding with chitosan dressings, Dialysis and transplantation, 2006 pp. 1-6.

According to a very common technique for obtaining hemostasis on an AVF, a compress, pad or cotton is placed at the puncture site and manual pressure is applied on the compress or cotton. One or more adhesive strips then hold the compress or cotton in place after release of the manual pressure.

The applicant has found that this common technique poses several difficulties.

In dialyzed patients having an AVF with strong blood flow, the effect of the anticoagulants is such that the bleeding time at the puncture site can be up to 45 minutes after the dialysis session (see document WO 03/099143 page 1 lines 11 to 20). Maintaining pressure on the AVF for such a long time is tedious and tiring. People suffering from neurodegenerative diseases (Parkinson, parkinsonian syndrome, Alzheimer) may not be capable of maintaining manual pressure at the puncture site. This is also true of patients who are agitated, depressed, epileptic, have chorea, or have convulsions for various reasons.

The stoppage of bleeding at the puncture or injection site must be checked regularly, which requires lifting the compress or cotton. Checking in this way can cause a spray of blood, with risk of contamination of healthcare personnel, or even a hemorrhage, particularly when the patient is agitated, or for dialyzed patients who must compress the venous access and arterial access of the AVF at the same time. Bleeding can occur during the compression of the puncture site, necessitating a compress change because the presence of blood conceals the puncture site. Regular checking for the stoppage of bleeding by partial or total removal of the compress may or may not cause the adhesion of the hemostatic compress on the puncture site, with a risk of detachment of the platelet plug.

Document US 2004/0092999 describes a latex elastic bracelet on which a rubber or latex hemispheric part is glued, the bracelet serving as tourniquet before venous puncture, and facilitating hemostasis after puncture, the rubber part compressing the puncture site through a compress. The rubber part described in this prior document makes it possible to avoid the application of manual pressure at the puncture site. However, the use of said bracelet does not make it possible to avoid the risks of blood spray or hemorrhage when the compression part is lifted to check the puncture site. The dressings described in documents WO 99/08723 and US 2005/0256438 have the same disadvantages. The same is true for the AVF compressive dressing described in document WO 03/099143.

Document U.S. Pat. No. 5,891,074 describes a compressive dressing comprising an absorbent polymer foam placed facing the puncture or injection site. The absorbent polymer foam is, for example, a polyurethane foam marketed by the company Avitar under the brand name Hydrasorb®. As a variant, a part made of spring steel or of a polymer material such as polycarbonate, polyethylene, or polyurethane is placed in direct contact with the skin. The dressing described in document U.S. Pat. No. 5, 891,074, like very many of the dressings proposed in the prior art, seek to apply pressure automatically at the puncture or injection site, instead of manual application.

Beyond the hemostatic agents cited in document US 2006/0155235, known from the prior art are a large number of chemical agents promoting hemostasis (made from gelatin, collagen, cellulose oxide, or chitosan). Bachtell et al. (Treatment of dialysis access puncture wound bleeding with chitosan dressings, Dialysis & Transplantation, November 2006) describe the use of a hemostatic dressing marketed by the company HemCon [sic: HenCon]. The dressing makes it possible to reduce the compression time on AVFs to a few minutes in order to obtain hemostasis. The dressings proposed by the company HenCon have been widely used in emergency contexts. Such dressings, for example marketed under the brand name HenCon Chito-Flex®, contain hemostatic agents (derived from chitosan, see http://www.fda.gov). These dressings have the disadvantage of concealing the puncture site so that it cannot be visually monitored while compressing the cutaneous bleeding point.

The present inventor has developed a dressing for arteriovenous fistula enabling the post-dialysis bleeding time to be reduced significantly (see Boulanger et al, Evaluation of post-puncture bleeding time of arteriovenous fistulas with Iris® bandage, J Vasc Access 2014 pp. 102-107). This dressing is marketed under the brand name Iris, and it makes it possible to reduce the post-dialysis compression time of arteriovenous fistulas.

The present inventor has found that some patients still have prolonged post-dialysis bleeding times, in spite of the use of the best available techniques. Prolonged post-dialysis bleeding is a frequent and severe complication, which affects the quality of life of patients and extends the time of dialysis sessions; medical personnel being particularly stressed at the end of a dialysis session.

The invention seeks to provide a solution to the problems presented above.

In particular, the invention seeks to propose a device and method for dressing a puncture or injection site, said device not having the disadvantages of those that were previously known, and ensuring a high degree of protection against AEBs, hemorrhages and bacterial contamination, in particular, but not exclusively, for dialysis vascular access.

To that end, according to a first aspect, a dressing is proposed for a puncture or injection site, comprising a sheet of micro-perforated polymer material, a first face of said sheet being covered with a pressure-sensitive adhesive, the opposite face of said sheet being non-adhesive, the dressing comprising a strip of material attached to the non-adhesive face of the sheet of polymer material, said strip of material being compressible and impermeable.

During the movement of removing a needle from a puncture or injection site, in particular at the end of dialysis, the application of the adhesive sheet on said puncture or injection site and around said site can lead to the formation of a direct passage of air between the adhesive sheet and the patient's skin, said direct passage being the imprint of the volume occupied by the needle. The application of manual pressure on the strip of compressible and impermeable material enables the re-closing of this direct passage, and makes it possible to prevent the creation of a depression in said passage, during the complete removal of the needle.

Advantageously, the strip of compressible and impermeable material forms a frame. Said arrangement makes it possible to use the dressing in various orientations.

According to various implementations, the dressing has the following characteristics, which may or may not be combined:
the strip of compressible and impermeable material is of expanded polymer, or a cellulose-based non-woven material, or made of polyethylene vinyl acetate;
the strip of compressible and impermeable material is of polyethylene foam;
the sheet of micro-perforated polymer material is transparent, translucent or semi-transparent;
the sheet of micro-perforated polymer material is made of polyethylene provided with an acrylic adhesive;
the sheet of micro-perforated polymer material is provided with perforations of a diameter of less than 0.5 mm;
the density of perforations of the sheet of micro-perforated polymer material is on the order of 100 per square centimeter.

According to a second aspect, a perfusion, drainage or catheterization kit is proposed comprising a dressing such as the one presented above, and a needle, a cannula, a catheter, or a drain.

According to a third aspect, the use of a dressing as presented above is proposed for dressing of arteriovenous fistula (AVF).

Other objects and advantages of the invention will be seen from the following description of embodiments. This description will be made with reference to the appended drawings in which:

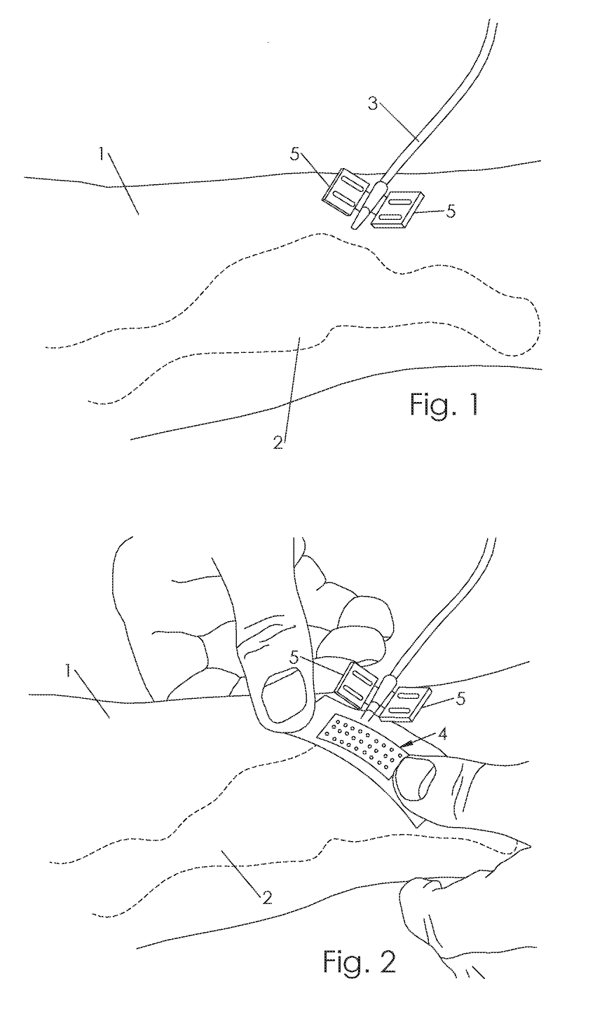

FIG. 1 is a view in perspective of the forearm of a patient having an AVF, a butterfly needle being in place in said AVF;

FIG. 2 is a view similar to FIG. 1, showing a first step of the method;

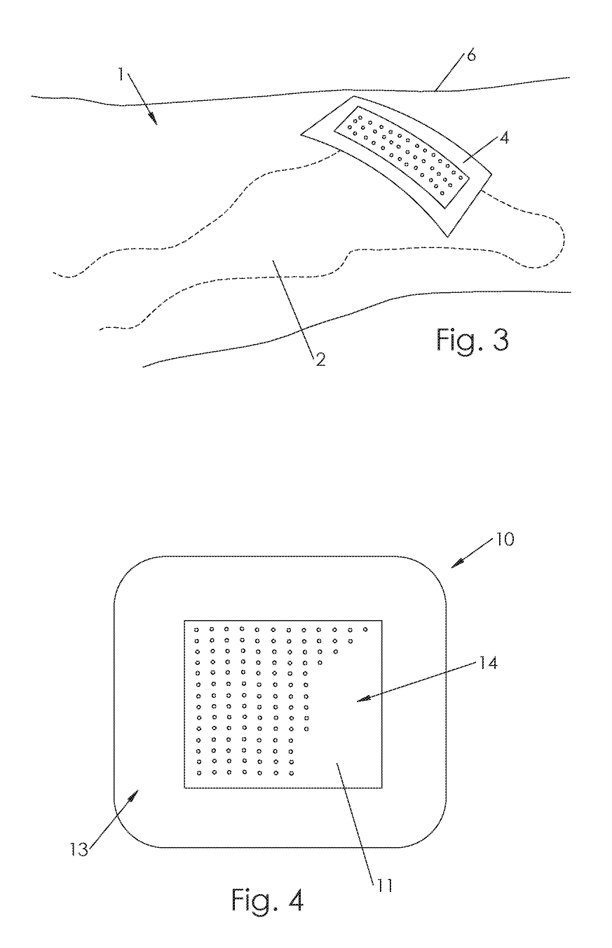

FIG. 3 is a view similar to FIGS. 1 and 2, showing a second step of the method;

FIG. 4 is a top view of a dressing according to one embodiment;

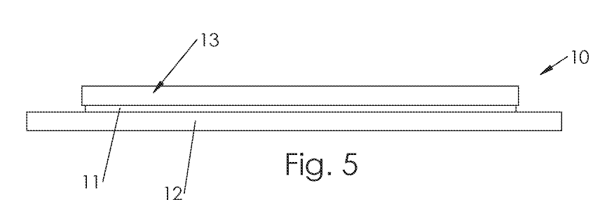

FIG. 5 is a side view of the dressing of FIG. 4.

Reference is now made to FIGS. 1 to 3.

Represented in FIG. 1 is the forearm 1 of a patient, having an AVF. For purposes of simplification, only one needle is still in place in said AVF. As a person skilled in the art will understand, the procedure described with reference to the figures is applied successively to the venous needle as well as the arterial needle.

In the appended figures, the needle is a butterfly type needle. However, it is understood that the method described also concerns needles without wings, or catheters (for example Cathlon®) or cannulas.

The AVF, first indication vascular access and the most widespread for dialysis patients, causes the appearance of aneurysmal areas 2, visible in the figures, the skin being highly deformed near the AVF. AVFs are often not very long, so that the two needles, artery and vein, must be placed close to each other. The use of holding tape can cause repeated irritation of the skin, promoting excoriations that are particularly susceptible to bacterial contamination. The fistulas can be kept active for many years, and the skin of some dialysis patients is not supple, and is thin and fragile because of age.

In a first step, represented in FIG. 2, the needle 3 is removed and the adhesive strip 4 is placed at the puncture site. Said adhesive strip 4 is transparent, semi-transparent or translucent.

Advantageously, said adhesive strip 4 is permeable to fluids, for example micro-perforated.

Advantageously, said adhesive strip 4 includes hemostatic compounds, for example mixed into the adhesive.

In a third step, represented in FIG. 3, a compress can be applied with pressure against the adhesive strip 4, level with the puncture site, the needle 3 being completely removed.

The pressure on the compress can then be released and a visual inspection of the hemostasis can be done. It should be noted that the adhesive strip 4 has not been removed, so that the puncture site remains covered during the visual inspection for hemostasis. The risks of AEB are therefore eliminated, along with the risks of bleeding related to removal of the platelet plug.

An adhesive strip, for example of the kind marketed under the brand name Tegaderm®, can then be placed on top of the compress, or on top of the adhesive strip 4.

The implementation of said method makes it possible to greatly reduce the compression time at the puncture sites of AVFs.

The applicant's experience shows that the mechanisms leading to this very significant reduction in compression time could be as follows.

In a first phase, after positioning the adhesive strip covering the puncture site, the blood leaving the puncture site does not flow beneath the adhesive strip 4 and remains confined. Said confinement phase is of variable length of time, depending on the hydration state of the total blood: the more the patient is in hyper-hydration, the longer this phase is. The length of this first phase is increased by taking anti-vitamin K, or in the presence of a platelet anomaly. The length of this first phase is also increased when the AVF is the location of a stenosis or when the puncture of the AVF is performed on an aneurysmal area or close to the anastomosis. The duration of this first phase can be reduced by exerting gentle pressure on the anastomosis for about two minutes.

In a second phase, shorter than the first one, the blood is strained by passing through the adhesive strip 4, which is permeable to fluid, and the serum is absorbed by the compress. The concentration of formed elements increases in the blood present at the puncture site, because of the straining and said absorption of the serum. The viscosity of the blood present at the puncture site increases. The perforations of the adhesive strip 4 are progressively occluded by the viscous blood.

The implementation of the method also makes it possible to greatly reduce the risks of resumption of bleeding.

The applicant's experience shows that the mechanisms leading to this reduction of the risk of resumption of bleeding could be the following.

The confinement and straining of the blood flowing from the puncture site leads to the creation of scabs of limited extent. When the compress is removed, the risk of detachment of these scabs is therefore reduced.

The sterile transparent semi-permeable adhesive strip 4 protects the vascular puncture site and leaves it visible at any time without risk of blood spray or the compress sticking directly to the puncture site.

Reference is now made to FIGS. 4 and 5 illustrating one embodiment of a dressing, enabling the use of the method described with reference to FIGS. 1 to 3.

The dressing 10 represented in FIGS. 4 and 5 comprises a first part 11 forming said adhesive strip 4. Advantageously a peel-off sheet 12 covers the adhesive face of said first part 11. In one implementation the peel-off sheet 12 is thin, for example on the order of 0.05 mm, siliconized polyester.

Advantageously, the adhesive strip 4 is semi-permeable and allows the diffusion of at least certain compounds of the blood, from the puncture site towards a compress placed facing the adhesive strip 4.

In one advantageous implementation, the adhesive strip is produced from flexible polymer material, or coated fabric, and is advantageously completely transparent, translucent or semi-transparent.

The flexibility of the dressing enables it to follow the contours of the skin, including near the aneurysmal areas of the AVFs. Advantageously, the material forming the adhesive strip, and if needed, all of the dressing 10, is stretchable at least in one longitudinal direction, and more advantageously still, in both longitudinal and transverse directions. The dressing 10 is thus even more adaptable to the different curvatures of the patient's body.

The perforation of the adhesive strip 4 is advantageously produced by implementing a method comprising the following steps:
application of glue on the face of the film before being made adhesive, for example a polyethylene (PE) film, particularly a low-density PE film 60 μm thick, the adhesive being acrylic-based, with a thickness of about 30 μm;
application of a liner covering the glued face;
perforation of the film, for example by hot needling, in a square- or diamond-shaped pattern, in one or two passes, the density of holes being advantageously on the order of 110 per square centimeter;
removal of the protective film and application of a peel-off strip.

Advantageously, the perforations intended for the straining are micro-perforations obtained without removal of material, for example by needling.

Advantageously, the adhesive strip 4 is elastic and the perforation is done with the adhesive strip under tension so that the micropores are substantially closed after release of the tension of the strip. When the dressing is placed on the patient, the manual stretching of the adhesive strip 4 causes the opening of said micropores.

The dressing comprises a frame 13 of flexible material. The frame can be square, rectangular or oval in shape. Advantageously, the frame 13 is made of foam, for example polyethylene foam. In other implementations, the frame 13 is made of a cellulose-based non-woven material, or polyethylene vinyl acetate-based material.

Before its removal, the needle occupies a space between the skin and the adhesive strip 4, said space forming a short path for passage of blood leaving the puncture site. The present inventor has found that to eliminate this risk, it is advantageous to apply manual pressure on the frame 13, above the needle while it is being withdrawn.

Advantageously, the frame 13 and the adhesive strip 4 do not include latex, avoiding allergic reactions to said compound.

By way of example, the dressing 10, when viewed from above, is substantially rectangular with rounded edges, of a length equal to about 40 mm and a width equal to about 35 mm. The frame 13 defines a substantially square central window 14, of about 20 mm per side. The thickness of the frame 13 is on the order of 1.5 to 2 mm.

The dressing 10 that has just been described has numerous advantages.

The present inventor has found that the risks of accident by exposure to blood were greatly reduced during the disconnection of the venous and arterial lines at the end of a dialysis session.

Indeed, in most patients only a drop of blood appeared and trickled at the point of passage of the needle. This very small amount of blood was absorbed by a compress pressed on top of the dressing, the compression time being a few dozen seconds. For a few rare patients, for example stenosis of the AVF or abnormal heart rate, the flow of the blood can occur by slight jet, but this flow is broken up by the presence of the micro-perforated adhesive strip. The dressing allows a visual monitoring of any prolonged bleeding, without risk of spray.

The present inventor has found that the compression times at the end of a dialysis session were greatly reduced.

The frame 13 can also participate in protection of the puncture or injection site against pressure and shocks, maintaining a space between the skin and the clothing of the patient. If needed, a compress can be maintained in place in the window 14 defined by the frame 13.

1. A dressing (10) for puncture or injection site, comprising a sheet of micro-perforated polymer material, a first face of said sheet being covered with a pressure-sensitive adhesive, the opposite face of said sheet being non-adhesive, characterized in that it comprises a strip of material attached to the non-adhesive face of the sheet of polymer material, said strip of material being compressible and impermeable.

2. The dressing (10) for puncture or injection site according to claim 1, wherein the strip of compressible and impermeable material forms a frame (13).

3. A dressing (10) for puncture or injection site, comprising a strip of compressible and impermeable material of expanded polymer, or a cellulose-based non-woven material, or made of polyethylene vinyl acetate.

4. The dressing (10) for puncture or injection site according to claim 3, wherein the strip of compressible and impermeable material is of polyethylene foam.

5. The dressing (10) for puncture or injection site according to claim 1, wherein the sheet of micro-perforated polymer material is transparent, translucent or semi-transparent.

6. The dressing (10) for puncture or injection site according to claim 5, wherein the sheet of micro-perforated polymer material is of polyethylene provided with an acrylic adhesive.

7. The dressing (10) for puncture or injection site according to claim 1, wherein the sheet of micro-perforated polymer material is provided with perforations of a diameter of less than 0.5 mm.

8. The dressing (10) for puncture or injection site according to claim 7, wherein the density of perforations of the sheet of micro-perforated polymer material is on the order of 100 per square centimeter.

9. A perfusion, drainage or catheterization kit comprising a dressing (10) as presented in claim 1, and a needle, a cannula, a catheter, or a drain.

10. A method of using a dressing (10), according to claim 1, comprising dressing an arteriovenous fistula AVF.
